(19) United States
(12) Patent Application Publication
(10) Pub. No.: US 2025/0195163 A1
(43) Pub. Date: Jun. 19, 2025

(54) ROBOT-ASSISTED IMAGING AND SERVOING FOR INTRAOPERATIVE GUIDANCE

(71) Applicant: Worcester Polytechnic Institute, Worcester, MA (US)

(72) Inventors: Songbai Ji, Westborough, MA (US); Xihan Ma, Worcester, MA (US); Haichong Zhang, Shrewsbury, MA (US); Arno Sungarian, Shrewsbury, MA (US)

(21) Appl. No.: 18/981,464

(22) Filed: Dec. 14, 2024

Related U.S. Application Data
(60) Provisional application No. 63/610,230, filed on Dec. 14, 2023.

(51) Int. Cl. A61B 34/32; A61B 34/20; A61B 90/00

(52) U.S. Cl. CPC A61B 34/32; A61B 2034/2057; A61B 2034/2065; A61B 2090/374; A61B 2090/3762; A61B 2090/378

(57) ABSTRACT

An intraoperative guidance approach for an image guided surgical robot performs intraoperative image registration and guidance based on a surgical target. A surgical imaging modality, such as US (ultrasound) or PA (photoacoustic), establishes a frame of reference based on a preoperative base imaging modality such as CT (computed tomography) or MRI (magnetic resonance imaging). A position reference of a surgical target, such as a tumor or neural structure, is established by the surgical imaging modality from the base imaging modality, computed based on a dense scan of the surgical region. Successive scans in the surgical image modality provide real-time guidance for a robotically actuated surgical instrument or probe based on the surgical target. An iteration of successive scans allows intramodal servoing concurrent with surgical instrument actuation towards a moving surgical target when a fixed surgical position in the frame of reference cannot be assured.

### RELATED APPLICATIONS

This patent application claims the benefit under 35 U.S.C. § 119(e) of U.S. Provisional Patent App. No. 63/610,230, filed Dec. 14, 2023, entitled “ROBOT-ASSISTED IMAGING AND SERVOING FOR INTRAOPERATIVE GUIDANCE,” incorporated herein by reference in entirety.

### STATEMENT OF FEDERALLY SPONSORED RESEARCH AND DEVELOPMENT

This invention was developed, at least in part, with U.S. Government support under contract No. DP5 OD028162, awarded by the National Institute for Health (NIH). The Government has certain rights in the invention.

### BACKGROUND

Despite the potential to improve surgical accuracy and patient outcomes, imaging based, robotic surgical guidance has yet to be widely adopted, largely because of challenges and limitations in patient registration. Neurological based procedures, such as spinal and cranial procedures, rely on precise manipulations. In spinal procedures, accumulated mobility between multiple rigid vertebrae prohibits the use of skin-affixed fiducials. Instead, spinal registration requires the surgeon to identify, expose, and localize anatomical landmarks within the surgical field, which often involves a substantial investment of time and effort that result in increased risk of morbidity to patient under general anesthesia.

### SUMMARY

An intraoperative guidance approach for an image guided surgical robot performs intraoperative image registration and guidance based on a surgical target. A surgical imaging modality, such as US (ultrasound) or PA (photoacoustic), establishes a frame of reference based on a preoperative base imaging modality such as CT (computed tomography) or MRI (magnetic resonance imaging). A position reference of a surgical target, such as a tumor or neural structure, is established by the surgical imaging modality from the base imaging modality, computed based on a dense scan of the surgical region. Successive scans in the surgical image modality provide real-time guidance for a robotically actuated surgical instrument or probe based on the surgical target. An iteration of successive scans allows intramodal servoing concurrent with surgical instrument actuation towards a moving surgical target when a fixed surgical position in the frame of reference cannot be assured.

Configurations herein are based, in part, on the observation that medial imaging technology is often employed for surgical procedures to locate surgical targets or treatment locations and minimize invasive measures needed for surgical intervention. Unfortunately, conventional approaches to medical imaging hardware suffers from the shortcoming that it can be bulky and expensive, and not well suited to concurrent use during surgical procedures. MRI (magnetic resonance imaging) requires a large magnetic coil to surround the imaging region, limiting access and causing problems for use of ferrous metals. CT (computed tomography) approaches are also not well suited for operating environments. US (ultrasound) and PA (photoacoustic) are rather compact and low cost, but can be problematic to obtain a continual imaging reference of a surgical target which may not remain completely stationary during the procedure, or require radioactive contrasting agents for registering images with a common frame of reference.

Accordingly, configurations herein substantially overcome the shortcomings of conventional imaging to provide interoperative navigation in a surgical environment using an imaging modality adapted for interoperative navigation as a robotic instrument traverses a surgical region towards a non-stationary surgical target. Surgical targets, such as tumors, growths, skeletal and neural structures may be subject to movement from an unanesthetized patient or surgical manipulation during a procedure. An interoperative navigation tracks and registers successive images for computing a position of the surgical target for engagement with a surgical instrument or probe. Intermodal analysis performs a translation of an imaged position in a frame of reference shared by the imaging modality and the surgical actuator to follow movement of the surgical target and corresponding actuation. In this manner, a series of images obtained during the surgical procedure are registered with the frame of reference of the surgical target for following movement and performing responsive actuation for attaining the surgical target by the surgical instrument.

In further detail, the disclosed system, method and apparatus provides a method for controlling a robotic element in a surgical region based on imaging transformation by establishing a frame of reference of a surgical target, and receiving an initial position reference indicative of a position of the surgical target from a surgical imaging modality within the frame of reference. A robotic guidance server actuates a robotic instrument based on the position of the surgical target in the frame of reference, and receives subsequent position references within the frame of reference. The server repositions the robotic instrument based on an intraoperative modality analysis of the subsequent position reference with the position reference in an iterative manner for attaining the surgical target.

### BRIEF DESCRIPTION OF THE DRAWINGS

The foregoing and other features will be apparent from the following description of particular embodiments disclosed herein, as illustrated in the accompanying drawings in which like reference characters refer to the same parts throughout the different views. The drawings are not necessarily to scale, emphasis instead being placed upon illustrating the principles of the invention.

FIG. 1 is a context view of a surgical and imaging environment suitable for use with configurations herein;

FIG. 2 is a process flow of intraoperative navigation in an imaging modality in the surgical environment of FIG. 1;

FIG. 3 shows intraoperative motion compensation in the surgical environment of FIGS. 1 and 2;

FIGS. 4A-4C show registration for intraoperative navigation of a moving surgical object as in FIG. 1; and

FIG. 5 is a flowchart of image and surgical manipulations as in FIG. 2.

### DETAILED DESCRIPTION

Configurations herein depict a system of robot-assisted, intuitive, and radiation-free image guidance to provide accurate intraoperative navigation for medical procedures such as surgery and radiotherapy. In particular use cases described below, spinal/vertebrae correction and intercranial malignancies are described as examples of potential implementation.

The disclosed approach exhibits a registration and guidance system that 1) uses a robotical mechanical driving system to avoid conventional cumbersome optical tracking, and avoids the line-of-sight limitations with the latter; 2) the robotic arm can be equipped with a range of intraoperative imaging modalities, such as (but not limited to) ultrasound and stereovision, that will be low-cost and radiation free compared to current fluoroscopy and CT imaging that are costly and also pose radiation safety concerns; and 3) robot-assisted intraoperative imaging can be acquired frequently on-demand, which, together with low-cost and radiation-free, will significantly broaden the use of image-guidance in surgery and radiotherapy, and maximize patient outcomes. The system is applicable whenever there is significant tissue deformation after preoperative images are acquired.

In conventional approaches, surgical navigation systems are significantly underutilized due to cumbersome and error-prone reliance of the radiographic imaging as it relates to real time mapping of the surgical anatomy. Conventional approaches provide no reliable real time image guidance for intracranial guidance as contents of the skull experience significant location shift upon elevating a cranial flap. Further, no reliable markers are available to distinguish normal brain from malignant tissue during real time resection. Intraoperative MRI is prohibitive in terms of intraoperative requirements, both in terms of access and sterility.

It would be beneficial to establish pathologic resection margins through high resolution microscope sampling. The disclosed approach provides accurate geographic sampling through robot assisted surface mapping.

Photoacoustic capabilities of the robotic system provide, with high confidence, real time distinction of normal versus abnormal tissue which will assist in surgical resection of tumors. The system also delivers secondary safety overlay by preventing human errors such as vascular and neural injury due to anatomic variations or misplacement of hardware, therefore eliminating surgeon fatigue and decreasing operative time. Consequently, this leads to improved patient outcomes and satisfaction due to decrease of morbidity and mortality, hospital stay and time under anesthesia.

However, most navigation systems and robotic platforms are underutilized due to cumbersome and bulky equipment, heavy reliance on intraoperative CT and fluoroscopy, registration errors, non-intuitive software programs and heavy reliance on wifi availability. Therefore, the majority of robotic systems currently available are used as a marketing tool of the companies that provide surgical instrumentation especially in orthopedic and spine surgery.

Similarly, surgical navigation systems for spinal and neurological procedures are significantly underutilized. Despite the potential to improve surgical accuracy and patient outcomes, image guidance has not been widely adopted in spine surgery, largely because of challenges and limitations in patient registration. Accumulated mobility between multiple rigid vertebrae prohibits the use of skin-affixed fiducials for assistance in robotic guidance. Instead, spinal registration requires the surgeon to identify, expose, and localize anatomical landmarks within the surgical field, which often involves a substantial investment of time and effort that results in increased risk of morbidity to patient under general anesthesia. Further, the typical one-time registration at commencement of surgery does not compensate for intervertebral motion due to a change in patient posture between preoperative scans and intraoperative intervention. In addition, common optical tracking suffers line-of-sight limitations that could negatively impact workflow in the operating room (OR). As a result, only 11% of spine surgeons in North America and Europe have been reported to use image guidance routinely, despite the well-recognized advantages of this technology in improving surgical accuracy, potential for facilitating complex surgery, and reducing radiation exposure.

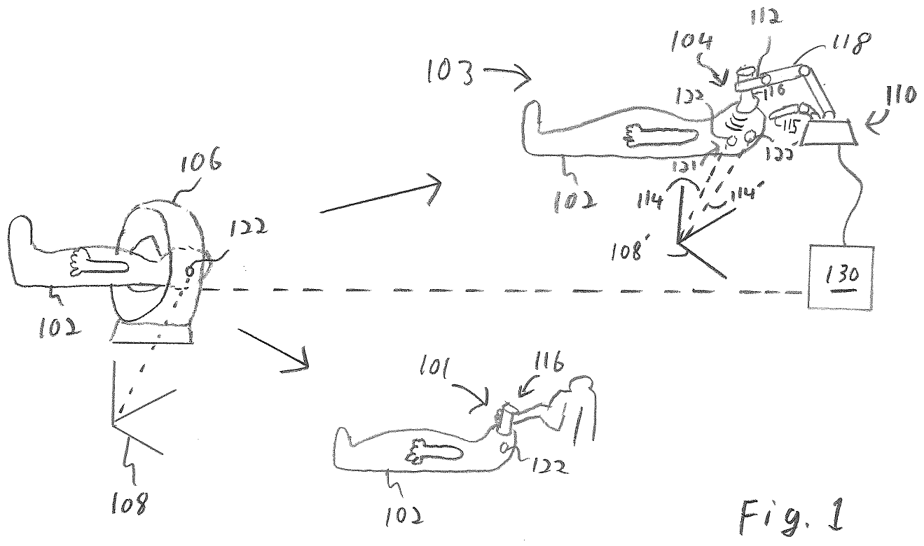

FIG. 1 is a context view of a surgical and imaging environment 100 suitable for use with configurations herein. Referring to FIG. 1, the disclosed method for controlling a robotic element 110 in a surgical region 104 based on imaging transformation includes establishing a frame of reference 108 of a surgical target 122 diagnosed in a patient 102. The frame of reference 108 is typically from a robust but non-portable imaging modality such as an MRI 106 or CT. Conventional approaches pursue manual US or PA imaging 101 with guidance from the initial MRI provided frame of reference 108. In the disclosed approach 103, a robotic actuator 112 of the robotic element 110 receives an initial position reference 114 indicative of a position 121 of the surgical target 122 from a surgical imaging modality within the frame of reference 108′, transformed from the MRI based frame 108. Typically this is an US probe 116 engaged by the robotic actuator 112. The robotic element 110 actuates a robotic instrument such as the US probe 116 based on the position 121 of the surgical target 122 in the frame of reference 108′. During the surgical procedure, an image server 130 receives a subsequent position reference 114′ within the frame of reference 108′ and repositions the robotic instrument (probe 116) based on an intraoperative modality analysis of the subsequent position reference 114′ with the position reference 114, thereby accommodating movement of the patient or surgical target as the surgery progresses.

To maximize the clinical benefits of spine image-guidance, it is preferable to develop more efficient, effective, and accessible image guidance techniques so that to substantially increase its adoption. Configurations herein propose a robot-assisted intraoperative ultrasound (riUS) for spine image-guidance by registering riUS with preoperative CT (pCT) on-demand throughout the spinal procedure. The novel advantages include (1) the use of real-time, radiation-free, low-cost, non-invasive, and minimally user-dependent riUS in place of the costly fluoroscopy or intraoperative CT (iCT) that also pose radiation concerns; (2) the elimination of time-intensive and cumbersome fiducial-based spine registration with handsfree riUS; and (3) the use of a mechanically driven robotic arm 118 to offer high tracking accuracy without suffering the line-of-sight limitations with conventional optical tracking system so that to improve surgical workflow.

Ongoing advancement in imaging modalities and inter/intra modality registration establishes the foundation for the disclosed US-based navigation and image-guidance in spine surgery. The approach herein depicts two important distinctions and improvements: (1) a robot-assisted platform with much reduced footprint to eliminate the conventional and cumbersome optical tracking that suffers the “line-of-sight” limitations and has also contributed to the slow adoption of image-guidance in spine, despite the well-recognized clinical benefits. Thus, this effort has the potential to significantly expand the technique adoption in spinal and other procedures; (2) a unique level-wise registration utilizes intra-modality US-US registration for servoing (plus a one-time registration between dense US and CT at surgery start), which is expected to improve over inter-modality US-CT registration by others.

The result is a registration-updated CT (uCT) for image navigation by (1) establishing an active sensing end-effector (A-SEE) to localize surgical region of interest for continuous and handsfree riUS image acquisitions; and (2) compensating for intervertebral motion in riUS-pCT registration using a continuous robot servoing strategy based on intramodality US-US registration to maintain accurate level-wise registration throughout the procedure, by iteratively receiving and analyzing a series of successive US images, or “slices.”

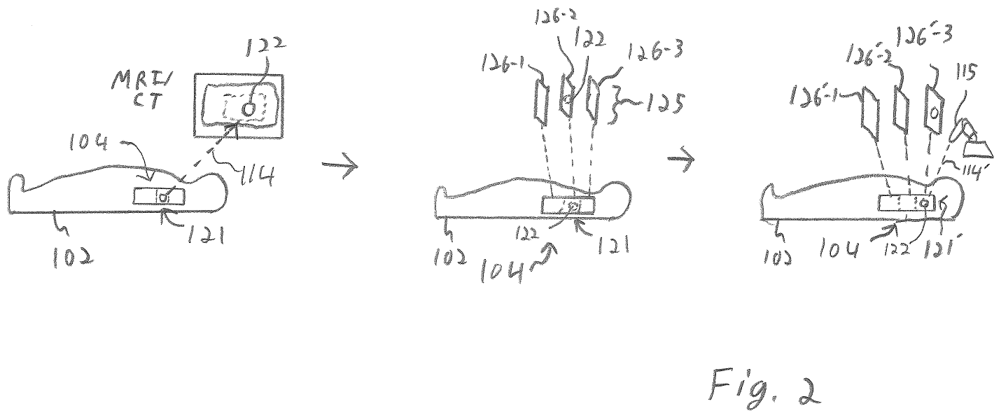

FIG. 2 is a process flow of intraoperative navigation in an imaging modality in the surgical environment of FIG. 1. Referring to FIGS. 1 and 2, in a typical workflow of a surgical procedure utilizing the disclosed approach, a patient undergoes an MRI or CT imaging for establishing the frame of reference 108 using a base imaging modality (MRI or CT) applied to a surgical region 104 of a patient. As the base imaging modality is generally unsuitable for a surgical (operating room) environment, a more portable US modality is used. An image server 130 registers the position reference 114 with the surgical imaging modality (US) within the frame of reference 108 of the base imaging modality, where the base imaging modality is a different imaging modality than the surgical imaging modality defining the position 121 and the subsequent position reference 114′. Various imaging modalities may be performed, however in the example configuration the base imaging modality is one of CT or MRI and the surgical imaging modality is one of US or PA.

After establishing the frame of reference 108 from an imaging of a surgical region by the base imaging modality, and determining the position 121 of the surgical target 122 based on the frame of reference 108, the image server 130 transforms the position of the surgical target to the frame of reference 108′ of the surgical imaging modality. As shown in FIG. 2, identifying the position of the surgical target involves receiving a dense scan 125 of a surgical region 104 including the surgical target 122 from the surgical imaging modality, and registering the dense scan 125 with the frame of reference 108 established by the base imaging modality. In other words, transforming the US imaged location in the same frame of reference of the MRI based on imaged features common to both the base imaging modality and the surgical imaging modality; alternatively an inserted fiducial common to both the base imaging modality and the surgical imaging modality may be employed. This allows the US to guide a surgical instrument 115 based on the initial dense scan 125.

One aspect to be computed by the image server 130 for registration accounts for the base imaging modality in a 3 dimensional medium and the surgical imaging modality returning a two dimensional US plane representation of the surgical region 104. In the example shown, US image “slices” 126-1 . . . 126-3 (126 generally) are shown, however an actual scan would likely return many more US images 126. It can be further seen that the surgical target 122 is shown in the location corresponding to image 126-2.

As the surgical procedure progresses, the image server 130 receives a series of subsequent position images 126′-1 . . . 126′-3, such that each of the subsequent position references may be indicative of a different position 121′ of the surgical target 122. The image server 130 reconstructs the two dimensional representations 126′ for computing the position reference 114′ for the surgical target 122 in the frame of reference 108′. The position 121′ of the surgical target 122 based on the successive position reference 114′ is indicative of a different position than the initial position reference 114, at position 121. The successive position reference 114′ is in a location corresponding to position image 126′-3, having moved from the position 121 depicted by position image 126-2. Thus the successive images 126 are registered based on the initial frame of reference 108 so as to be able to compute movement as occurring from features or structures depicted in the successive images 126′

Given that preoperative CT (pCT) or MRI alone, without the costly and ionizing iCT, fluoroscopy or O-arm, can offer navigation directly with comparable accuracy, the use of image-guidance as disclosed herein is viable to expand for maximizing the clinical impact of image-guidance and patient outcomes. However, efficient and accurate image registration as described above is crucial, yet not possible with simple skin-affixed fiducials, which may not adequately reflect movement. In open procedures, spinal registration involves substantial effort from the surgeon to identify, expose, and localize anatomic landmarks. The one-time registration at the start of surgery does not compensate for intervertebral motion resulting from patient postural changes (e.g., supine in pCT acquisition vs. prone in surgery) or intraoperative intervention, itself. These negatives perceptions make it difficult to adopt image-guidance, which has subsequently created distrust in the value of image-guided approaches that has also slowed acceptance even for minimally invasive interventions. The use of subsequent position images overcomes this shortcoming.

The disclosed approach of FIG. 2 therefore depicts robot-assisted intraoperative ultrasound (riUS) co-registered with pCT to provide image guidance. Robotic assisted surgery has considerably expanded since its inception, offering potential to overcome physical and mental fatigue, hand tremor, difficulties with manual dexterity, and surgical precision, concurrently with low-cost, non-invasive, and radiation-free ultrasound (US). The result is automatic, accurate, and efficient vertebral level-wise (i.e., treating each level individually vs. all levels as a whole) registrations between riUS and pCT to generate registration-updated CT (uCT) that will enable on-demand image-guidance and navigation throughout the lumbar spinal procedure. Additionally, this allows comprehensively evaluate registration efficiency and accuracy with intraoperative CT (iCT) and bone-implanted fiducials to determine if riUS alone (with uCT), without costly and ionizing iCT, is capable of providing sufficient accuracy for image-guidance in lumbar spinal operations. Alternate configurations complement the established registration used to overlay with the skin surface view via the mounted RGB-D (Red-Green-Blue-Depth) camera for pedicle screw insertion guidance. It will also serve as the basis for a second companion co-robotic arm in future development, invoking the RGB-D camera for image-guidance with submillimeter accuracy.

In contrast to conventional approaches, the disclosed approach employs the dual-arm robotic US platform for spine image-guidance with an auto-aligning feature, which will enable precise control of the US probe 116 with repeatable procedures that are less user dependent. The robotic arm 118 offers motion tracking accuracy without the line-of-sight limitations with conventional optical tracking system used in existing surgical workflow. A robotic US platform with an active sensing end-effector (A-SEE) may be invoked to enable spontaneous normal positioning of probe relative to skin surface based on real-time distance sensing for US image acquisition, described further in copending U.S. patent application Ser. No. 18/381,510, filed Oct. 18, 2023, entitled “ROBOTIC ASSISTED IMAGING,” assigned to the assignee of the present application and incorporated herein by reference in entirety. Further, the level-wise registration may be combined with the novel A-SEE will provide automatic and adaptive image-guidance throughout the surgery. This avoids limitations with other competing co-robotic approaches that are either based on a specific scanning trajectory or a manual and one-time pre-plan. The handsfree and automatic riUS-pCT registration pipeline will eliminate time-intensive and cumbersome fiducial-based spine registration; thus, further streamlines the surgical workflow.

One approach to normal positioning relies on balancing between two diagonally paired sensors per rotation axis, which may be vulnerable to any sensor misreading or blockage. We will generalize the normal position estimation algorithm that suits different sensor arrangements and exhibits higher tolerance to sensor failures. The normal direction is defined as the normal vector of a local surface in F_{A-SEE}, where the US probe 116 touches the patient body. Considering m distance measurements, a set of 3D points p∈ [symbol] ^{m×3} on the patient body w.r.t F_{A-SEE} can be obtained. We form k local mesh triangles by sampling vertices from p. The normal vector of the contact surface n∈ [symbol] ³ is estimated through a weighted sum of the normal vectors of all local mesh triangles as equation (1) where n₍is the normal vector for each mesh triangle pointing inwards to the body; ω₍is the weight proportional to the area of each triangle. Under circumstances where some sensors stop working, the normal direction estimation can still be done as long as m≥3. The normal direction tracking aims to align the end-effector's approach vector (z-axis) a∈ [symbol] ³ to n. It is achieved by imposing angular velocities about the x-axis (ωₓ) and y-axis (ω_{y}) of F_{A-SEE} based on (2) where n_{xz} and n_{yz} are the projections of n in x-z and y-z plane; a_{xz} and a_{yz} are the projections of a in x-z and y-z plane; θ(·) calculates the angle between two vectors; K_{p,x} and K_{p,y} are constant gains.

$$n = \frac{{\sum }_{i = 1}^{k} {ω}_{i} {n}_{i}}{{\sum }_{i = 1}^{k}} , \qquad ( 1 )$$

$$[ \begin{matrix}{ω}_{x} \\ {ω}_{y}\end{matrix} ] = [ \begin{matrix}{K}_{p , x} & 0 \\ 0 & {K}_{p , y}\end{matrix} ] [ \begin{matrix}θ ( {n}_{yz} , {a}_{yz} ) \\ θ ( {n}_{xz} , {a}_{xz} )\end{matrix} ] \qquad ( 2 )$$

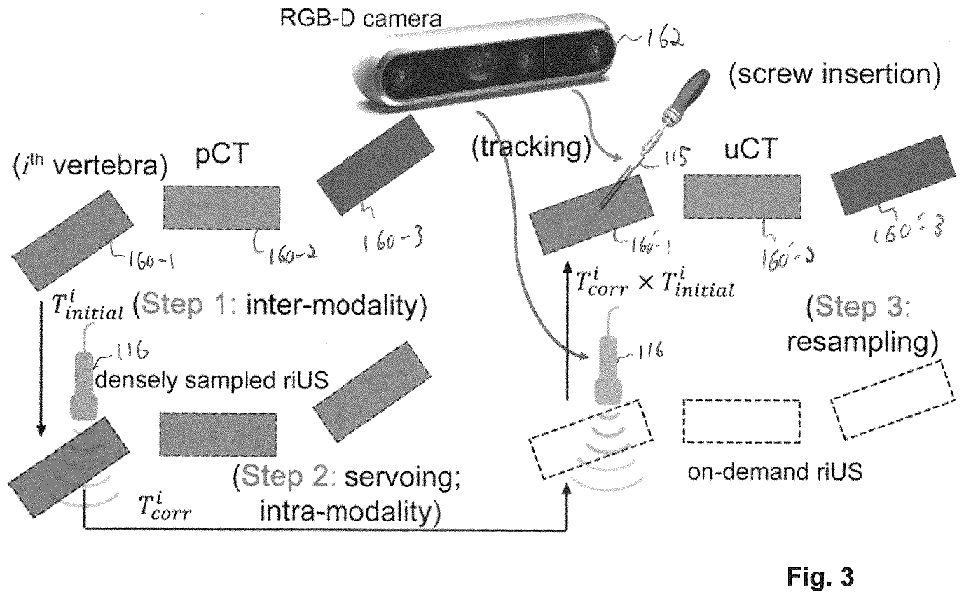

FIG. 3 shows intraoperative motion compensation in the surgical environment of FIGS. 1-2. Intraoperative motion compensation through riUS/pCT registration & US servoing includes 3 steps. Step 1 performs initial inter-modality (US/CT) level-wise registration. This receives densely sampled riUS (DS-riUS as in FIG. 3, for the spine or other surgical target, and will be used to establish a vertebral level-wise rigid riUS-pCT registration. An established approach is to apply forward and backward scan line tracing to CT and US, respectively, to extract vertebral posterior surface and then maximize their intensity cross-correlation. It achieves a TRE (Transformation-based Reconstruction Error) of 1.33-1.68 mm under 10 sec for pedicle screw insertion in porcine cadavers. This establishes the frame of reference using the base imaging modality (CT) applied to the surgical region 104 of the patient 102, and registers the position reference with the surgical imaging modality (US) within the frame of reference of the base imaging modality, therefore allowing precise and accurate placement in vertebrae 160-1 . . . 160-3 (160 generally) to a position of vertebrae 160′-1 . . . 160′-3 established by successive position images 126.

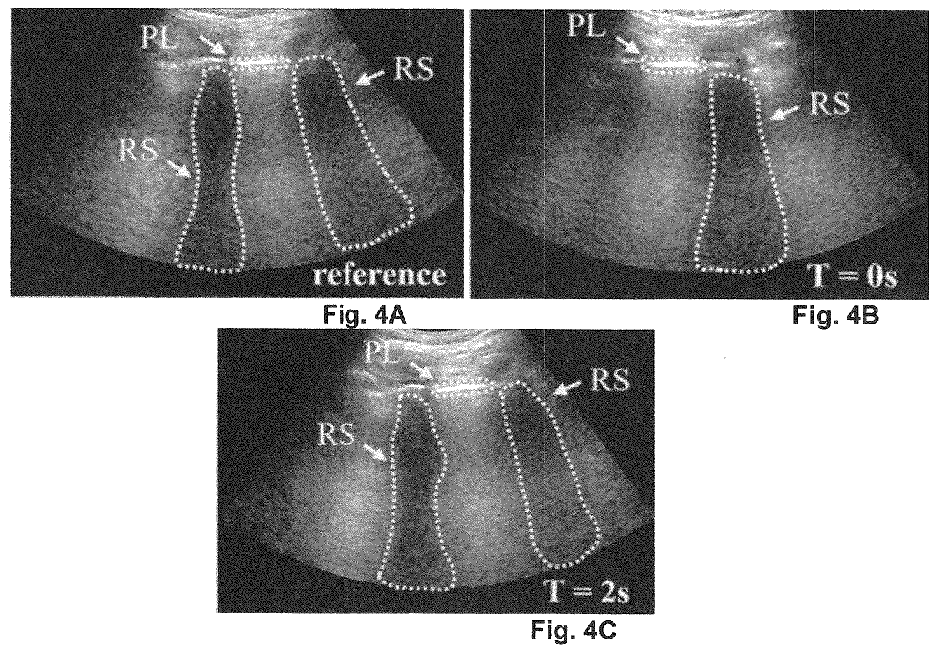

Step 2 of FIG. 3 involves autonomous probe positioning through riUS servoing to maintain accurate level-wise registration between on-demand riUS and DS-riUS via intra-modality registration. This refers to US-US repositioning of the robotic instrument based on an intraoperative modality analysis of the subsequent position reference with the position reference. FIGS. 4A-4C show registration for intraoperative navigation of a moving surgical object as in FIG. 1. Referring to FIGS. 4A-4C, and continuing to refer to FIGS. 1-3, FIG. 4A shows riUS servoing for a lung US application in a live human subject. The riUS system automatically identifies anatomical features (PL: plural line, RS: rib shadow.

After identifying the image containing spinal features (e.g., vertebrae), the probe pose adjusts to minimize the misalignment of the current vertebral visualization w.r.t to a reference pose based on US/CT registration in Step 1. First, to quantify the in-plane misalignment, we shift the reference image from the DS-riUS data in lateral (x) and axial (z) axis by Δx and Δz, respectively, and rotate around the elevational axis (y) by Δθ, shown in FIG. 4B. The misalignment is given by [Δx, Δz, Δθ] where the cross-correlation between the acquired (FCMRob) and reference (FCMRef) image is maximized. Next, the estimated in-plane misalignment, IPM (FCMRob, FCMRef), will be used to calculate the in-plane velocity command with respect to the image frame (equation 3) where Vx, z, θ is the velocity in translation along x and z and rotation around y; K_{p,US} is the control gain. During in-plane adjustment, the velocity imposed in z (denoted as v_{Z,s}) could conflict with the force compliant controller. A velocity fusion algorithm is therefore necessary to balance in-plane alignment and force compliance. The velocity fusion algorithm that yields the final z-axis velocity v₊ is formulated (equation 4), where α(·)∈[0, 1] is a scalar function that rapidly decreases as the instantaneous force error e_{f}[t] approaches to zero, whereas α(·)=0 for e_{f}[t]≤0. Intuitively, the algorithm prioritizes force compliance when the contact force is close to the desired value but allows in-plane alignment along z if the instantaneous contact force is small. Once the in-plane alignment is established, the probe will scan along the out-of-plane direction, Δy, to compensate for the elevational misalignment. FIGS. 4A-4C demonstrate an example of riUS servoing for a lung application in live humans (N=3). Within 2 s, residual probe pose error was reduced to <2 mm in translation and <2 degrees in rotation. Such results are equally applicable to the spinal procedures.

$${V}_{x , z , θ} = {K}_{p , US} \cdot IPM ( {FCM}_{Rob} , {FCM}_{Ref} ) , \qquad ( 3 )$$

$${v}_{z} = α ( {e}_{f} [ t ] ) {v}_{z , s} + ( 1 - α ( {e}_{f} [ t ] ) ) {v}_{z , f} , \qquad ( 4 )$$

Step 3 performs generation of registration-updated CT (uCT) to provide navigation using an image modality most familiar to surgeons that also reflects intraoperative anatomy for best image guidance. This provides repositioning of the robotic instrument based on an intraoperative modality analysis of the subsequent position reference with the position reference. The riUS servoing will adjust the position and orientation of the US probe to maintain an identical US acquisition throughout the surgery. Effectively, the inverse of the relative change in US probe 3D position and orientation dictates the spatial change of the sampled vertebra of interest. The resulting correction in spatial position/orientation w.r.t. the DS-riUS, Tⁱ_{corr}∈SE(3) will be concatenated to the initial riUS-pCT registration for the corresponding vertebra, Tⁱᵢₙᵢₜᵢₐₗ∈SE(3). A registration-updated CT (uCT) for the corresponding vertebra will then be generated by transforming the corresponding pCT (i.e., Tⁱ_{corr}×Tⁱᵢₙᵢₜᵢₐₗ) for guidance.

To generate uCT for the entire lumbar spine, the robotic arm will be programmed to automatically land to the adjacent vertebra 160 to perform riUS servoing. This process will generate a series of Tⁱ_{corr} for all the lumbar vertebrae involved. Together with the corresponding Tⁱᵢₙᵢₜᵢₐₗ, they would compensate for intervertebral motion of the multi-segment rigid-bodies. A 3D warping displacement field will then be generated for each pCT vertebral voxel, and an image volume resampling will be used to generate uCT, analogously to model-updated MR for the brain. To mitigate conflict between two adjacent vertebrae 160 at boundary, a box filter will be similarly invoked.

To provide intraoperative feedback for pedicle screw insertion trajectory, the preoperatively defined insertion plan (position and orientation) may be projected on the RGB-D camera 162 view based on the US probe rigid-body transformation. The screw/tool pose tracked by the camera 162 via an optical marker will be fused with the navigation to form feedback for insertion guidance, as shown in FIG. 3. At the same time, uCT will be fused with tracked tool trajectory to provide additional guidance.

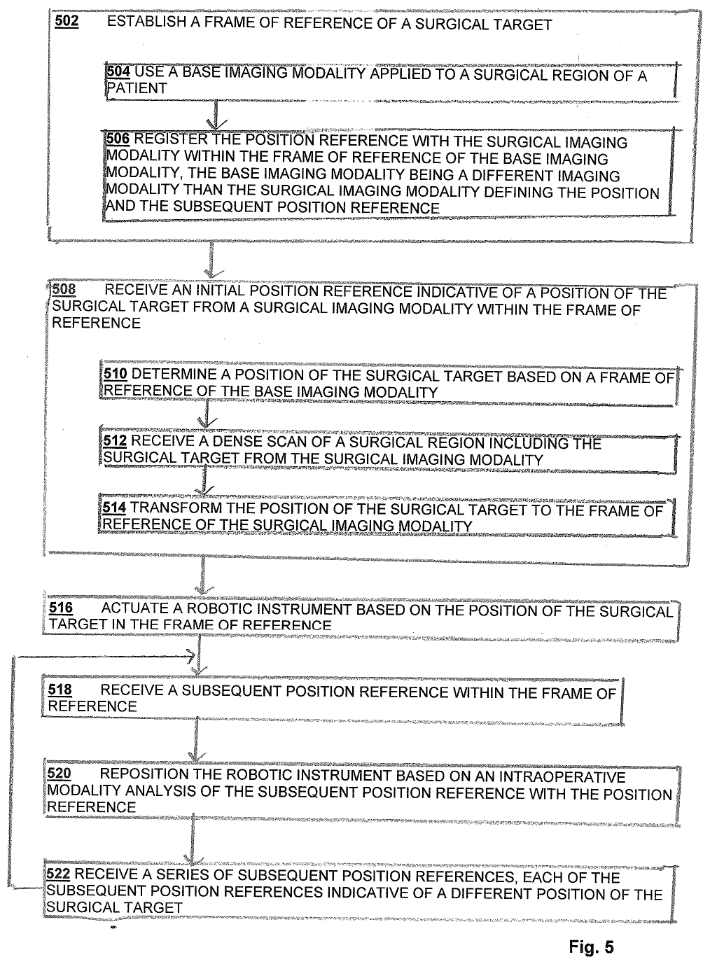

FIG. 5 is a flowchart of image and surgical manipulations as in FIG. 2. Referring to FIGS. 1-5, the method for controlling a robotic element in a surgical region based on imaging transformation includes, at step 502, establishing a frame of reference 108 of a surgical target 122. The initial imaging employs a base imaging modality applied to a surgical region of a patient 102, as depicted at step 504. The position reference with the surgical imaging modality is registered within the frame of reference 108 of the base imaging modality, as shown at step 106, where the base imaging modality is a different imaging modality than the surgical imaging modality defining the position and the subsequent position reference, typically an initial MRI followed by a US deep scan and successive US images 126. The image server 130 receives an initial position reference indicative of a position 121 of the surgical target 122 from a surgical imaging modality within the frame of reference, as depicted at step 508. This further includes determining the position 121 of the surgical target 122 based on a frame of reference 108 of the base imaging modality, as disclosed at step 510, and 512 a dense scan of a surgical region 104 including the surgical target 122 from the surgical imaging modality, as depicted at step 512.

The image server 130 then transforms the position of the surgical target 122 to the frame of reference of the surgical imaging modality, therefore merging or registering the US with the initial CT/MRI scan based on common features or fiducial.

The image server 130 then actuates the robotic instrument 115 based on the position of the surgical target 122 in the frame of reference 108′, as shown at step 516. As the instrument 115 advances, the robotically controlled US 116 returns a subsequent position reference 114′ within the frame of reference 108′ as depicted at step 518. The image server 130 then repositions the robotic instrument 115 based on an intraoperative modality analysis of the subsequent position reference 114′ with the position reference 114, as depicted at step 520. The US 116 is also robotically guided to follow the surgical instrument 115 while traversing the surgical region 104. The image server 130 iteratively receives a series of subsequent position references as depicted at step 522, such that each of the subsequent position references 114′ is indicative of a different position 121′ of the surgical target 122.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for controlling a robotic element in a surgical region based on imaging transformation, comprising:
establishing a frame of reference of a surgical target;
receiving an initial position reference indicative of a position of the surgical target from a surgical imaging modality within the frame of reference;
actuating a robotic instrument based on the position of the surgical target in the frame of reference;
receiving a subsequent position reference within the frame of reference; and
repositioning the robotic instrument based on an intraoperative modality analysis of the subsequent position reference with the position reference.

2. The method of claim 1 further comprising:
establishing the frame of reference using a base imaging modality applied to a surgical region of a patient; and
registering the position reference with the surgical imaging modality within the frame of reference of the base imaging modality, the base imaging modality being a different imaging modality than the surgical imaging modality defining the position and the subsequent position reference.

3. The method of claim 2 wherein the base imaging modality is one of CT (computed tomography) or MRI (magnetic resonance imaging) and the surgical imaging modality is one of a US (ultrasound) or PA (photoacoustic).

4. The method of claim 1 further comprising:
establishing the frame of reference from an imaging of a surgical region by a base imaging modality;
determining a position of the surgical target based on a frame of reference of the base imaging modality; and
transforming the position of the surgical target to the frame of reference of the surgical imaging modality.

5. The method of claim 2 further comprising:
identifying the position of the surgical target within the frame of reference;
receiving a dense scan of a surgical region including the surgical target from the surgical imaging modality;
registering the dense scan with the frame of reference established by the base imaging modality.

6. The method of claim 2 wherein the base imaging modality is a 3 dimensional medium and the surgical imaging modality returns a two dimensional representation of the surgical region.

7. The method of claim 6 further comprising reconstructing the two dimensional representation for computing the position reference for the surgical target in the frame of reference.

8. The method of claim 1 wherein the position of the surgical target based successive position reference is indicative of a different position than the initial position reference.

9. The method of claim 1 further comprising:
receiving a series of subsequent position references, each of the subsequent position references indicative of a different position of the surgical target.

10. The method of claim 2 further comprising registering the position reference of the surgical imaging modality with the base imaging modality based on imaged features common to both the base imaging modality and the surgical imaging modality.

11. The method of claim 2 further comprising registering the position reference of the surgical imaging modality with the base imaging modality based on an inserted fiducial common to both the base imaging modality and the surgical imaging modality.

12. The method of claim 2 further comprising employing an RGB-D camera for external positioning of the actuator in the surgical region; and
registering the RGB-D camera with the frame of reference.

13. A system for controlling a robotic element in a surgical region based on imaging transformation, comprising:
a base imaging modality configured for establishing a frame of reference of a surgical target;
an image server receiving an initial position reference indicative of a position of the surgical target from a surgical imaging modality within the frame of reference;
a robotic instrument configured for actuation based on the position of the surgical target in the frame of reference, and
a probe responsive to the surgical imaging modality for receiving a subsequent position reference within the frame of reference, the robotic instrument configured for repositioning the robotic instrument based on an intraoperative modality analysis of the subsequent position reference with the position reference.

14. The system of claim 13 wherein the image server establishes the frame of reference using the base imaging modality applied to a surgical region of a patient, and registering the position reference with the surgical imaging modality within the frame of reference of the base imaging modality, the base imaging modality being a different imaging modality than the surgical imaging modality defining the position and the subsequent position reference.

15. The system of claim 14 wherein the base imaging modality is one of CT (computed tomography) or MRI (magnetic resonance imaging) and the surgical imaging modality is one of a US (ultrasound) or PA (photoacoustic).

16. A computer program embodying program code on a non-transitory computer readable storage medium that, when executed by a processor, performs steps for implementing a method for method for controlling a robotic element in a surgical region based on imaging transformation, the method comprising:
establishing a frame of reference of a surgical target;
receiving an initial position reference indicative of a position of the surgical target from a surgical imaging modality within the frame of reference;
actuating a robotic instrument based on the position of the surgical target in the frame of reference;
receiving a subsequent position reference within the frame of reference; and
repositioning the robotic instrument based on an intraoperative modality analysis of the subsequent position reference with the position reference.
